(19) United States
(12) Patent Application Publication
(10) Pub. No.: US 2026/0034309 A1
(43) Pub. Date: Feb. 5, 2026

(54) Pre-filled Lock Syringe With Integrated Cap

(71) Applicant: Becton, Dickinson and Company, Franklin Lakes, NJ (US)

(72) Inventors: Shishir Prasad, Ramsey, NJ (US); Karthik MR, Bangalore (IN); Manish Kumar, Bengaluru (IN); Prasad Govindaraj, Coimbatore (IN); Balaji Kannan, Chennai (IN); Joseph Mummert, Ramsey, NJ (US); Matthew Biggers, Guttenberg, NJ (US); Brendan Hickey, Franklin Lakes, NJ (US); Ajinkya Joglekar, Edgewater, NJ (US)

(21) Appl. No.: 18/995,198

(22) PCT Filed: Aug. 2, 2023
(86) PCT No.: PCT/US23/29279

§ 371 Date: Jan. 16, 2025

(30) Foreign Application Priority Data
Aug. 17, 2022 (IN) 202211046843

(51) Int. Cl. A61M 5/315; A61M 5/31; A61M 5/34

(52) U.S. Cl. CPC A61M 5/31511; A61M 5/31501; A61M 5/347; A61M 2005/3118

(57) ABSTRACT

Disclosed herein is a pre-filled syringe system including a pre-filled syringe including a barrel including a proximal end, a distal end, and a sidewall extending between the proximal and distal end, the sidewall defining a chamber that contains a medical solution, a plunger rod, and a stopper attached to the distal end of the plunger rod and moveably received in the chamber, and a disinfecting cap coupled to the pre-filled syringe.

### CROSS-REFERENCE TO RELATED APPLICATION

The present application claims priority to Indian Provisional Application No. 20/221,1046843, entitled “Pre-Filled Lock Syringe with Integrated Cap”, filed Aug. 17, 2022, the entire disclosure of which is hereby incorporated by reference in its entirety.

### BACKGROUND

### Field of the Invention

The present disclosure relates generally to pre-filled syringes, and, more specifically, to pre-filled syringes for delivering lock solutions to catheters, where the pre-filled syringe includes a disinfecting cap as a part of the syringe.

### Description of Related Art

Flush syringes, scrubbing devices, lock syringes, and disinfecting caps are currently sold as separate products. These products need to be arranged bedside before the flushing of an IV catheter is needed. The use of caps for passive disinfection and maintaining a physical barrier has been a recent development that is still gaining access within clinical facilities and is often overlooked by clinicians. There is a need in the field to provide a comprehensive solution for medical procedures that utilize a catheter, to allow for adequate disinfecting and catheter flushing and locking.

### SUMMARY

Accordingly, provided are improved systems, devices, products, apparatus, and/or methods for integrating a pre-filled syringe with a disinfecting cap.

According to some non-limiting embodiments or aspects, provided is a pre-filled syringe system, comprising: a pre-filled syringe, comprising: a barrel comprising: a proximal end, a distal end, and a sidewall extending between the proximal and distal end, the sidewall defining a chamber that contains a lock solution; a plunger comprising a plunger rod; and a stopper attached to a distal end of the plunger rod and moveably received in the chamber of the housing of the syringe body; and a disinfecting cap coupled to the pre-filled syringe.

In accordance with an embodiment of the present invention, a pre-filled syringe includes a barrel including a proximal end, a distal end, and a sidewall extending between the proximal and distal end, the sidewall defining a chamber that contains a medical solution, a plunger rod having a proximal end and a distal end, a stopper attached to the distal end of the plunger rod and moveably received in the chamber, and a disinfecting cap releasably coupled to the pre-filled syringe. The disinfecting cap includes a housing defining an interior configured to hold a disinfecting solution.

In certain configurations, the plunger includes a thumb press at the proximal end of the plunger rod, and a socket positioned between the plunger rod and the thumb press of the plunger. The disinfecting cap is contained within the socket.

In other configurations, the plunger further includes a thumb press at the proximal end of the plunger rod, and a cap holder including a lock and positioned between the plunger rod and the thumb press. The disinfecting cap includes a pin, and the pin is configured to releasably couple with the lock such that the cap can be removed from the cap holder.

In other configurations, the plunger further includes a thumb press at the proximal end of the plunger rod, and a cap holder attached to the thumb press, with the disinfecting cap received within the cap holder.

In certain configurations, the barrel may include a flange at the proximal end thereof, the flange including one or more slots, and wherein the disinfecting cap includes a guide configured to be positioned within the one or more slots to releasably attach the disinfecting cap to the pre-filled syringe.

In still other configurations, the disinfecting cap includes a ring including a snap connection, wherein the ring is configured to be arranged around an outer surface of the barrel to releasably attach the disinfecting cap to the pre-filled syringe.

In other configurations, the barrel includes a flange at the proximal end of the barrel, the flange including a slot having one or more openings therein, and wherein the disinfecting cap includes a plate comprising at least one pin, wherein the plate is configured to be positioned within the slot such that the at least one pin is positioned within the at least one opening to releasably attach the disinfecting cap to the pre-filled syringe.

In certain configurations, the barrel includes a flange at the proximal end of the barrel, the flange having a recess including at least one slot, and wherein the disinfecting cap includes at least one rib extending axially on the outer surface of the disinfecting cap, such that the at least one rib is configured to be positioned within the at least one slot to releasably attach the disinfecting cap to the pre-filled syringe.

In certain other configurations, the barrel includes a flange at the proximal end of the barrel, and wherein the disinfecting cap includes a peel film that is heat sealed to the flange, thereby attaching the disinfecting cap to the pre-filled syringe.

The plunger rod may include an internal volume at the proximal end of the plunger rod, and a thumb press attached to the internal volume with a hinge, wherein the disinfecting cap is configured to be positioned within the internal volume of the plunger.

In certain configurations, the disinfecting cap is configured to be releasably attached to an inner surface of the thumb press.

In other configurations, the pre-filled syringe further includes a tether having a first end and a second end, wherein the first end is attached to the pre-filled syringe and the second end is attached to the disinfecting cap.

In other configurations, the plunger rod includes a thumb press at the proximal end of the plunger rod, wherein a peel film of the disinfecting cap is attached to the thumb press.

In still other configurations, the disinfecting solution includes an alcohol.

In certain configurations, the medical solution is a flush solution.

In other configurations, wherein the medical solution is a catheter lock solution.

In certain configurations, the barrel includes a luer connector at the distal end thereof.

In certain configurations, the pre-filled syringe further includes a scrubbing device releasably coupled to the luer, the scrubbing device includes a housing defining an interior configured to hold a disinfecting solution.

In still other configurations, the disinfecting solution comprises an alcohol.

### BRIEF DESCRIPTION OF THE DRAWINGS

Additional advantages and details are explained in greater detail below with reference to the exemplary embodiments that are illustrated in the accompanying schematic figures, in which:

FIG. 1 is a cross-sectional view of a prior art pre-filled syringe;

FIGS. 2A-2D are side and partial views of a non-limiting embodiment of a pre-filled syringe as described herein;

FIGS. 3A and 3B are side and partial views of a non-limiting embodiment of a pre-filled syringe as described herein;

FIGS. 4A-4D are side and partial views of a non-limiting embodiment of a pre-filled syringe as described herein;

FIGS. 5A-5D are side and partial views of a non-limiting embodiment of a pre-filled syringe as described herein;

FIGS. 6A-6C are perspective, side, and partial views of a non-limiting embodiment of a pre-filled syringe as described herein;

FIGS. 7A-7D are side and partial views of a non-limiting embodiment of a pre-filled syringe as described herein;

FIGS. 8A-8C are side and partial views of a non-limiting embodiment of a pre-filled syringe as described herein;

FIGS. 9A and 9B are side and partial views of a non-limiting embodiment of a pre-filled syringe as described herein;

FIG. 10A is a side view of a non-limiting embodiment of a pre-filled syringe as described herein;

FIG. 10B is a side view of a non-limiting embodiment of a pre-filled syringe as described herein.

FIG. 11 is a perspective view of a non-limiting embodiment of a pre-filled syringe as described herein;

FIG. 12 is a side view of a non-limiting embodiment of a pre-filled syringe as described herein; and

FIG. 13 is a perspective view of a non-limiting embodiment of a pre-filled syringe as described herein.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” As used in the specification and the claims, the singular form of “a,” “an,” and “the” include plural referents, such as unless the context clearly dictates otherwise. Additionally, Furthermore, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms.

Provided are improved systems and products for integrating a cap with a pre-filled syringe. Embodiments of the present disclosure may include a pre-filled syringe and a disinfecting cap that is integrated with the pre-filled syringe to form a pre-filled syringe system. The pre-filled syringe systems provided herein integrates a cap with the last product (e.g., pre-filled lock syringe) used in IV line access and allow for easy access to the cap after use of the pre-filled syringe while providing a physical barrier to contamination and helping to keep the IV line disinfected. The pre-filled syringe systems provided herein also enhances compliance to SASH procedures and maintain all important features of pre-filled lock syringes including the attachment with the hub and interaction with the users thumb while depressing the plunger rod.

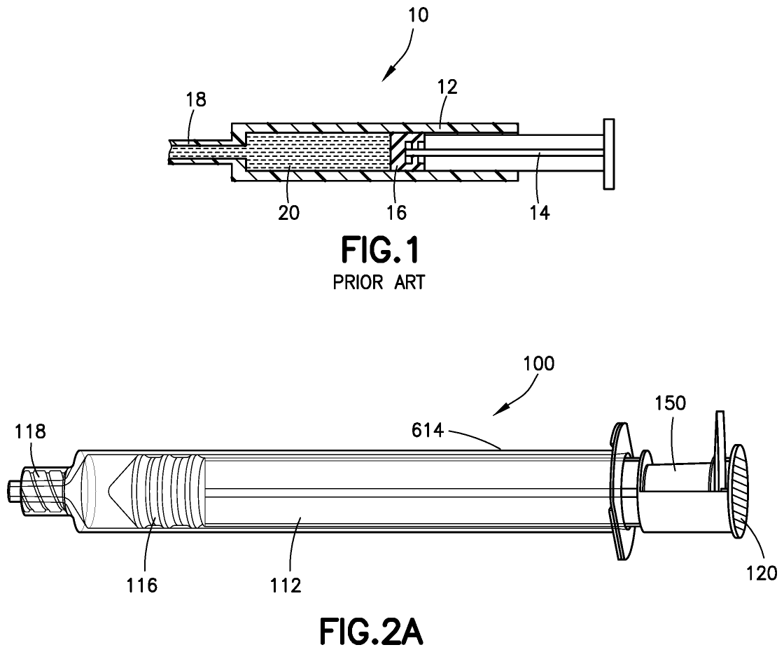

Referring to FIG. 1, FIG. 1 is a diagram of a pre-filled syringe 10. Pre-filled syringe 10 may be formed of various materials known in the art for a pre-filled syringe. For example, pre-filled syringe 10 may be formed of glass and/or plastic. In some non-limiting embodiments, pre-filled syringe 10 may be formed of polypropylene. In some non-limiting embodiments, pre-filled syringe 10 includes a barrel 12, plunger rod 14, stopper 16, and luer connection 18. Barrel 12, plunger rod 14, and tip cap (not illustrated) may be formed of various materials known for pre-filled syringes. For example, barrel 12, plunger rod 14, and the tip cap may be formed of glass and/or plastic. In some non-limiting embodiments, barrel 12, plunger rod 14, and the tip cap may be formed of polypropylene. In some non-limiting embodiments, plunger rod 14 may include a thumb press on the end of plunger rod 14 opposite of stopper 16. Barrel 12 and/or stopper 16 may be coated with suitable coatings as are known in the art, for example to reduce interactions between components contained the solution 20 of the pre-filled syringe, to reduce friction between components.

In some non-limiting embodiments, pre-filled syringe 10 contains medical solution 20. Medical solution 20 may include any solution and/or medication known in the art that is suitable for delivery with a pre-filled syringe. For example, pre-filled syringe 10 may contain a medication, a flush solution for a catheter, and/or a catheter lock solution, including, for example, those solutions described in U.S. Pat. Nos. 9,872,941 and 11,045,589, and/or U.S. Provisional Patent Application No. 63/223,191, the disclosures of which are hereby incorporated by reference in their entirety.

In some non-limiting embodiments, pre-filled syringe 10 is designed or configured to reduce or prevent instances of reflux of blood into a catheter at the conclusion of flushing and/or administration of a lock solution as described herein. In some non-limiting embodiments, pre-filled syringe 10 is configured such that the plunger rod 14 is shorter than a typical plunger rod, such that compression of the stopper 16 following infusion of lock solution 20 is substantially or completely prevented. In some non-limiting embodiments, the stopper 16 is designed or configured such that the nose of the stopper comes into contact with a distal end of the barrel, adjacent the luer connection 18, and blocks the opening, preventing vacuum and thus reflux of blood into the catheter.

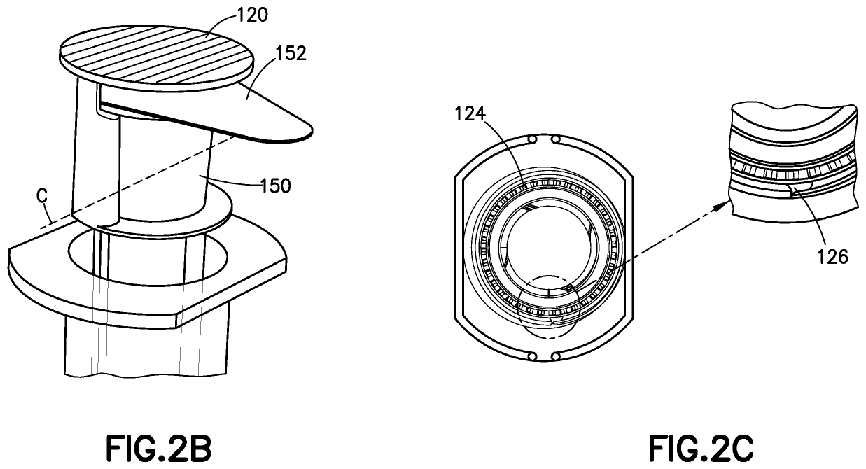

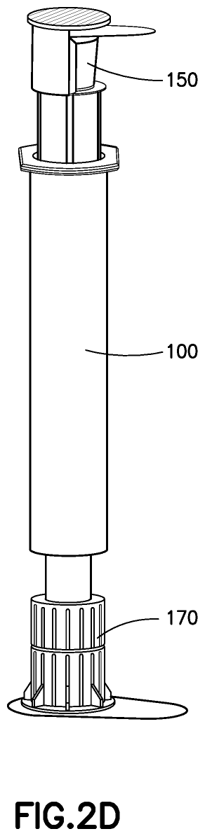

Referring now to FIGS. 2A-2D, shown are non-limiting embodiments of pre-filled syringe 100. In some non-limiting embodiments, pre-filled syringe 100 may include barrel 112, plunger rod 114, stopper 116, luer connection 118, and thumb press 120. In some non-limiting embodiments, pre-filled syringe 100 may be the same as pre-filled syringe 10 previously described, except that plunger rod 114 may be modified to integrate cap 150 as shown in FIGS. 2A-2D.

In some non-limiting embodiments, plunger rod 114 may include socket 124 which is a cut-out region of the plunger, distal of thumb press 120, which is configured to hold cap 150. In some non-limiting embodiments, plunger rod 114 is modified to accommodate cap 150 along central axis C. In some non-limiting embodiments, socket 124 may be semi-circular, such that socket 124 is open on one side to allow cap 150 to be removed, and, on the other side, includes a wall that connects thumb press 120 to the rest of plunger rod 114. In some non-limiting embodiments, socket 124 may include a tapered entry 126. Tapered entry 126 may be tapered inward to hold cap 150 in socket 124.

In some non-limiting embodiments, pre-filled syringe 100 includes cap 150. Cap 150 may be formed of various materials. For example, cap 150 may be formed of plastic. In some non-limiting embodiments, cap 150 may include peel film 152. Peel film 152 may be coupled to (e.g., attached, adhered, and/or the like) cap 150. In some non-limiting embodiments, peel film 152 may seal an internal volume of cap 150 and prevent contamination of the internal volume of cap 150. Cap 150 may contain, in interior thereof, one or more disinfecting solutions for disinfecting a luer connection on a catheter. In non-limiting embodiments, cap 150 includes one or more substrates, for example a porous substrate, impregnated with a disinfecting solution. Suitable disinfecting solutions are known to those of skill in the art, and may include various alcohols, such as isopropyl alcohol, for example 70% isopropyl alcohol. Cap 150 may further include a thread on an internal surface thereof, to allow for cap to be secured to a luer of a catheter.

In some non-limiting embodiments, cap 150 may be positioned within socket 124 of plunger rod 114. In some non-limiting embodiments, cap 150 may be held within socket 124 aided by tapered entry 126. In some non-limiting embodiments, peel film 152 may protrude radially out of socket 124.

Returning to FIGS. 2B and 2C, in some non-limiting embodiments, cap 150 may be removed from socket 124 of plunger rod 114. For example, peel film 152, and/or a flange arranged on cap 152, may protrude radially out of socket 124 and may be pulled such that cap 150 is dislodged from socket 124. Once cap 150 is dislodged from socket 124, cap 150 may be ready for use, as described above.

Referring to FIG. 2D, in some non-limiting embodiments, pre-filled syringe 100 may include scrubbing device 170. Scrubbing device 170 may be coupled to (e.g., attached to, connected to, and/or the like) pre-filled syringe 100. In some non-limiting embodiments, scrubbing device 170 may be connected to pre-filled syringe 100 via luer connection 118. Scrubbing device may be similar to cap 150, in that it may include a substrate, for example a porous substrate, impregnated with a disinfecting solution, where such solutions are known to those of skill in the art. In non-limiting embodiments, disinfecting solution may be an alcohol, such as isopropyl alcohol, for example 70% isopropyl alcohol. In use, scrubbing device 170 may be used to disinfect a luer of a catheter prior to delivery of a solution held within pre-filled syringe 110, and cap 150 may be used to provide long-lasting protection to catheter following delivery.

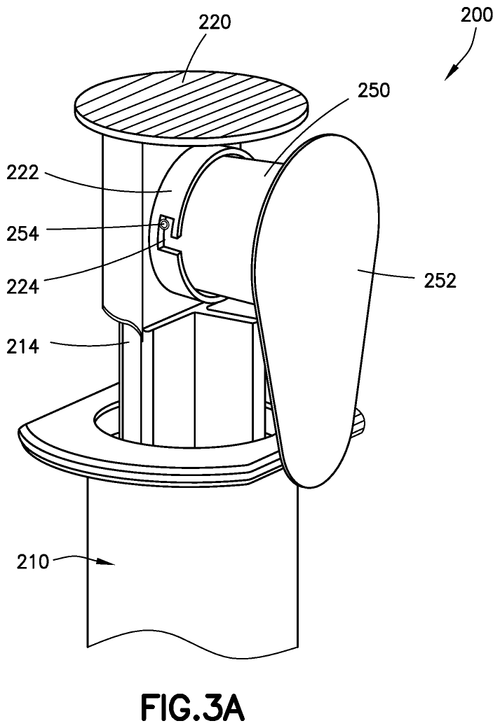

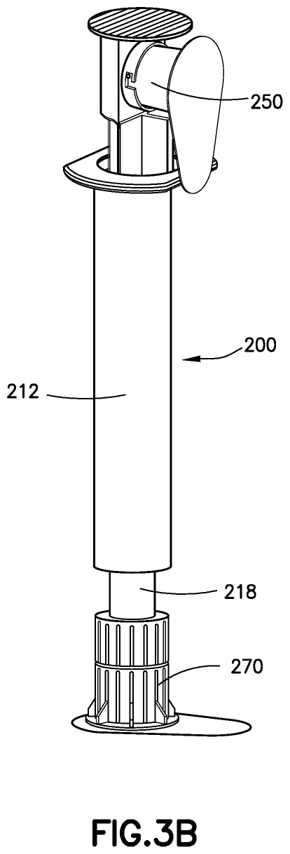

Referring to FIGS. 3A and 3B, shown are non-limiting embodiments of pre-filled syringe 200. In some non-limiting embodiments, pre-filled syringe 200 may include barrel 212, plunger rod 214, a stopper, luer connection 218, and thumb press 220. In some non-limiting embodiments, pre-filled syringe 200 may be the same as pre-filled syringe 10 previously described, except that plunger rod 214 may be modified to integrate cap 250 as shown in FIGS. 3A and 3B.

In some non-limiting embodiments, plunger rod 214 may include cap holder 222. For example, plunger rod 215 may include cap holder 222 located adjacent to thumb press 220. In some non-limiting embodiments, cap holder 222 may protrude out of plunger rod 214 and may be shaped to hold cap 250. In some non-limiting embodiments, cap holder 222 may include lock 224. For example, cap holder 222 may include lock 224 that is configured to receive lock pin 254 of cap 250. In some non-limiting embodiments, lock 224 may be a bayonet lock.

In some non-limiting embodiments, pre-filled syringe 200 includes cap 250. In some non-limiting embodiments, cap 250 may be the same as cap 150 previously described. In some non-limiting embodiments, cap 250 may include pin 254. For example, cap 250 may include pin 254 that is configured to be coupled with lock 224 of cap holder 222. In some non-limiting embodiments, pin 254 may be a bayonet pin. In some non-limiting embodiments, cap 250 includes peel film 252. For example, peel film 252 may be the same as peel film 152 previously described.

In some non-limiting embodiments, cap 250 may be positioned at least partially within cap holder 222. For example, cap 250 may be positioned at least partially within cap holder 222 such that peel film 252 faces away from plunger rod 214. In some non-limiting embodiments, pin 254 of cap 250 may be coupled to lock 224 of plunger rod 214 to secure cap 250 to plunger rod 214. For example, pin 254 of cap 250 may be coupled to lock 224 of plunger rod 214 via a bayonet lock connection. In some non-limiting embodiments, cap 250 may be removed from cap holder 222 by uncoupling pin 254 and lock 224. For example, cap 250 may be uncoupled from cap holder 222 may uncoupling a bayonet connection between pin 254 and lock 224. In such an example, pin 254 and lock 224 may be uncoupled by rotating cap 250 and then pulling cap 250 away from cap holder 222.

Referring to FIG. 3B, in some non-limiting embodiments, pre-filled syringe 200 may include scrubbing device 270, for example a scrubbing device as described above. In some non-limiting embodiments, scrubbing device 270 may be coupled to (e.g., attached to, connected to, and/or the like) pre-filled syringe 200. In some non-limiting embodiments, scrubbing device 270 may be connected to pre-filled syringe 210 via luer connection 218.

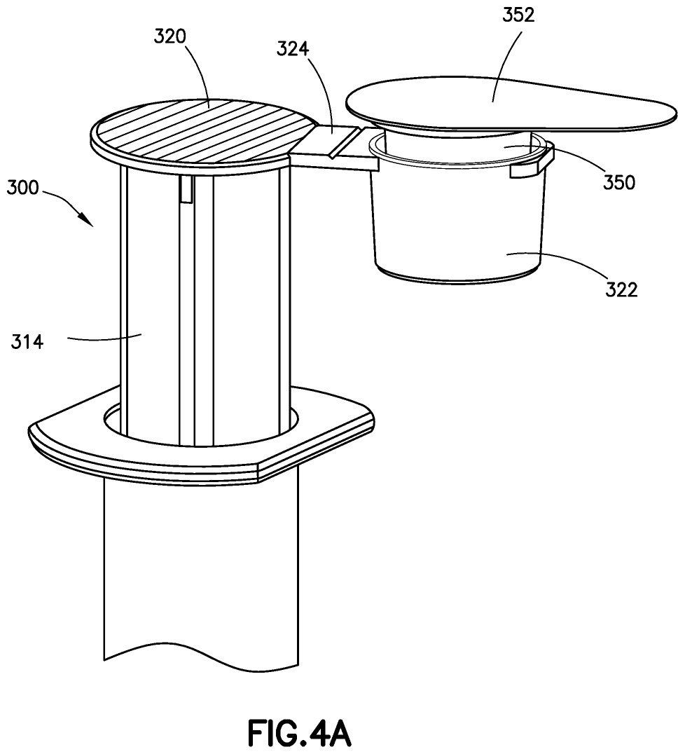

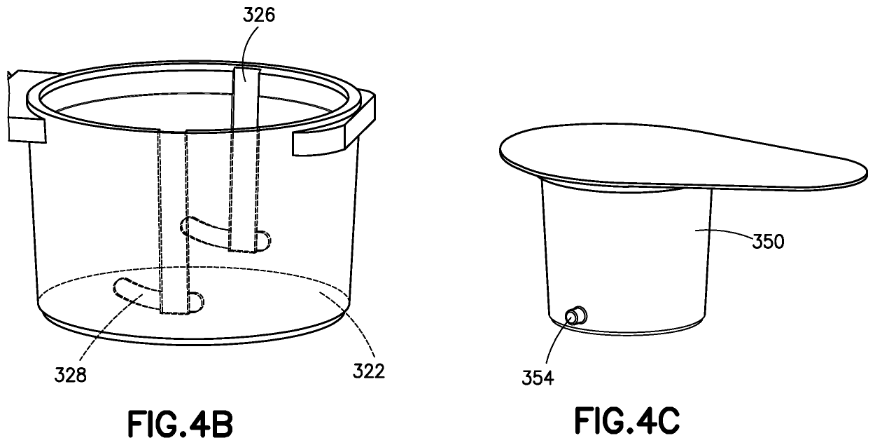

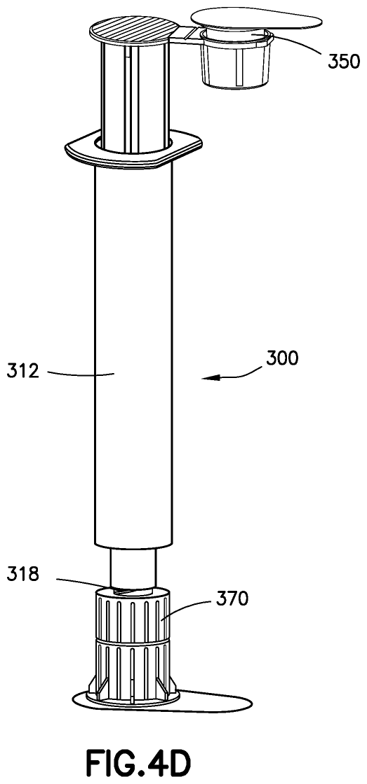

Referring to FIGS. 4A-4D, shown are non-limiting embodiments of pre-filled syringe 300. In some non-limiting embodiments, pre-filled syringe 300 may include barrel 312, plunger rod 314, a stopper, luer connection 318, and thumb press 320. In some non-limiting embodiments, pre-filled syringe 300 may be the same as pre-filled syringe 10 previously described, except that plunger rod 314 may be modified to integrate cap 350 as shown in FIGS. 4A-4D.

In some non-limiting embodiments, plunger rod 314 may include cap holder 322. Cap holder 322 may be connected to plunger rod 314 by a hinge support 324. In some non-limiting embodiments, hinge support 324 may connect cap holder 322 to thumb press 320. In some non-limiting embodiments, cap holder 322 may include an internal volume that is configured to hold cap 350. In some non-limiting embodiments, cap holder 322 may include at least one sliding slot 326. For example, cap holder 322 may include at least one sliding slot 326 that extends vertically along the inner surface of the cap holder 322. In some non-limiting embodiments, cap holder 322 may include at least one bayonet slot 328. For example, cap holder 322 may include at least one bayonet slot that is in communication with the at least one sliding slot 326 and extends horizontally along the inner surface of the cap holder 322 from the bottom of the at least one sliding slot 326. In some non-limiting embodiments, the at least one sliding slot 326 and the at least one bayonet slot 328 may be configured to be coupled to at least one pin 354 of cap 350.

In some non-limiting embodiments, pre-filled syringe 300 includes cap 350. In some non-limiting embodiments, cap 350 may be the same as cap 150 previously described. In some non-limiting embodiments, cap 350 may include at least one pin 354. For example, cap 350 may include at least one pin 354 that is configured to be coupled with the at least one sliding slot 326 and the at least one bayonet slot 328 of cap holder 322. In some non-limiting embodiments, cap 350 may be removed from cap holder 322 by rotating cap 350 and pulling cap 350 vertically out of cap holder 322, such that the at least one pin 354 moves through the at least one bayonet slot 328 and up through the at least one sliding slot 326. In some non-limiting embodiments, cap 350 includes peel film 352. For example, peel film 352 may be the same as peel film 152 previously described.

Referring to FIG. 4D, in some non-limiting embodiments, pre-filled syringe system 300 may include scrubbing device 370. In some non-limiting embodiments, scrubbing device 370 may be the same as scrubbing device 170 previously described. In some non-limiting embodiments, scrubbing device 370 may be coupled to (e.g., attached to, connected to, and/or the like) pre-filled syringe 310. In some non-limiting embodiments, scrubbing device 370 may be connected to pre-filled syringe 300 via luer connection 318.

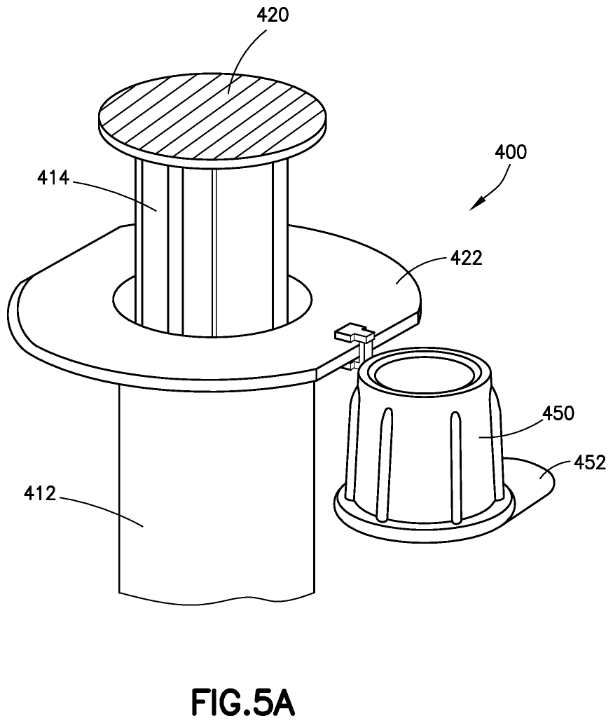

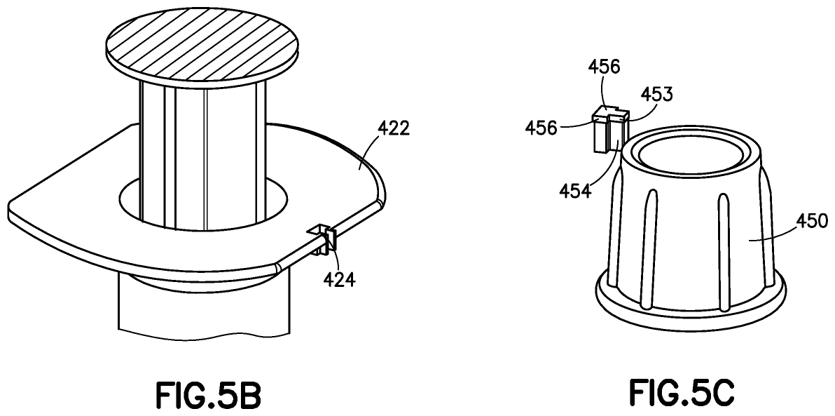

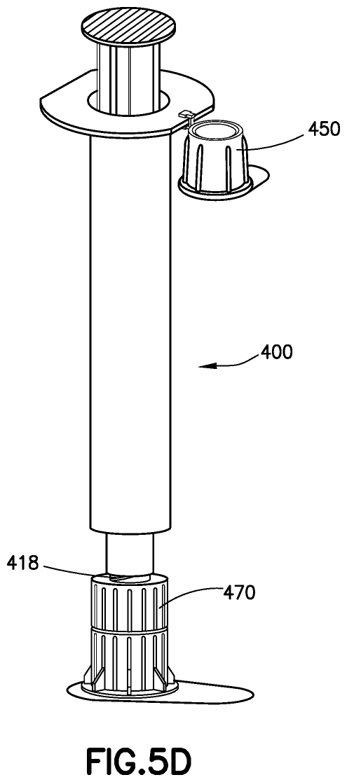

Referring to FIGS. 5A-5D, shown are non-limiting embodiment of pre-filled syringe 400. In some non-limiting embodiments, pre-filled syringe 400 includes barrel 412, plunger rod 414, a stopper, luer connection 418, and thumb press 420. In some non-limiting embodiments, pre-filled syringe 400 may be the same as pre-filled syringe 10 previously described, except pre-filled syringe 400 includes modified flange 422 as shown in FIGS. 5A-5D.

In some non-limiting embodiments, flange 422 may include slot 424. For example, flange 422 may include slot 424 that extends vertically through flange 422. In some non-limiting embodiments, slot 424 may include a “T” cross-section extending vertically through flange 422. In some non-limiting embodiments, slot 424 may be configured to be coupled to guide 454 of cap 450.

In some non-limiting embodiments, pre-filled syringe 400 includes cap 450. In some non-limiting embodiments, cap 450 may be the same as cap 150 previously described. In some non-limiting embodiments, cap 450 may include guide 454. For example, cap 450 may include guide 454 that is attached to the outer surface of cap 450. In some non-limiting embodiments, guide 454 may be configured such that it includes a cross-sectional “T” shape, formed by member 453 and protrusion(s) 456 (as shown in FIG. 5C, for example). For example, guide 454 may include a “T” cross-section that corresponds to a “T” cross-section of slot 424 on flange 422. In some non-limiting embodiments, guide 454 may include one or more protrusions 456. For example, guide 454 may include protrusion 456 that extends out from the surface of member 453. In some non-limiting embodiments, guide 454 may be configured to be coupled to slot 424 on flange 422. For example, guide 454 may be configured to be coupled to slot 424 on flange 422 and protrusion 456 may aid in coupling guide 454 to slot 424 on flange 422. In some non-limiting embodiments, cap 450 includes peel film 452. For example, peel film 452 may be the same as peel film 152 previously described.

Referring to FIG. 5D, in some non-limiting embodiments, pre-filled syringe 400 may include scrubbing device 470. In some non-limiting embodiments, scrubbing device 470 may be the same as scrubbing device 170 previously described. In some non-limiting embodiments, scrubbing device 470 may be coupled to (e.g., attached to, connected to, and/or the like) pre-filled syringe 410. In some non-limiting embodiments, scrubbing device 470 may be connected to pre-filled syringe 410 via luer connection 418.

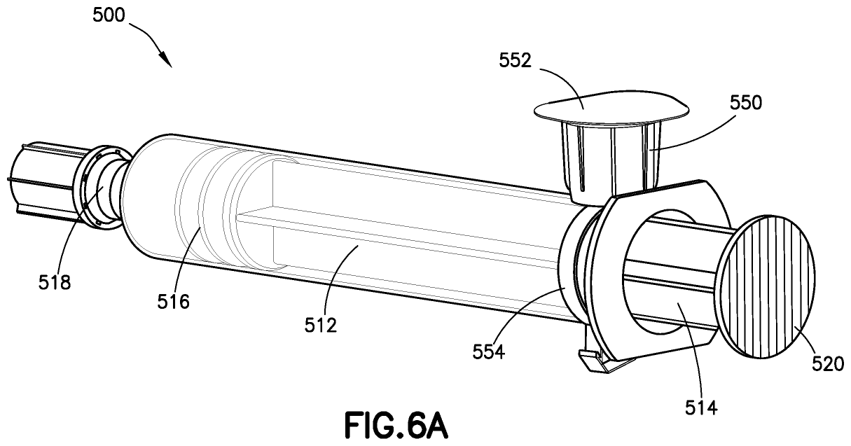

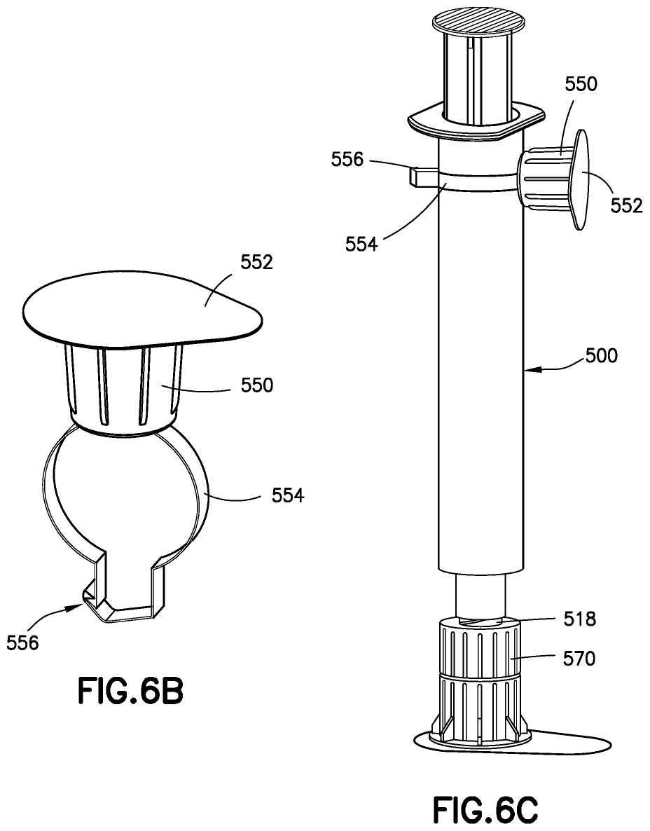

Referring to FIGS. 6A-6C, shown are non-limiting embodiments of pre-filled syringe 500. In some non-limiting embodiments, pre-filled syringe 500 may include barrel 512, plunger rod 514, stopper 516, luer connection 518, and thumb press 520. In some non-limiting embodiments, pre-filled syringe 500 may be the same as pre-filled syringe 10 previously described.

In some non-limiting embodiments, pre-filled syringe 500 includes cap 550. In some non-limiting embodiments, cap 550 may be the same as cap 150 previously described. In some non-limiting embodiments, cap 550 may include ring 554. For example, cap 550 may include ring 554 that is attached to outer surface of cap 550, for example at a proximal end (opposite of film 552) thereof. In some non-limiting embodiments, ring 554 may include snap connection 556. Snap connection 556 may allow for ring 554 to be opened and closed. In some non-limiting embodiments, cap 550 includes peel film 552. For example, peel film 552 may be the same as peel film 152 previously described.

In some non-limiting embodiments, cap 550 may be coupled to pre-filled syringe 500, such as coupled to barrel 512. For example, ring 554 may be positioned around barrel 512 to couple cap 550 to pre-filled syringe 510. In some non-limiting embodiments, cap 550 may be removed from pre-filled syringe 510 by opening snap connection 556 and pulling cap 550 away from barrel 512.

Referring to FIG. 6C, in some non-limiting embodiments, pre-filled syringe 500 may include scrubbing device 570. In some non-limiting embodiments, scrubbing device 570 may be the same as scrubbing device 170 previously described. In some non-limiting embodiments, scrubbing device 570 may be coupled to (e.g., attached to, connected to, and/or the like) pre-filled syringe 510. In some non-limiting embodiments, scrubbing device 570 may be connected to pre-filled syringe 510 via luer connection 518.

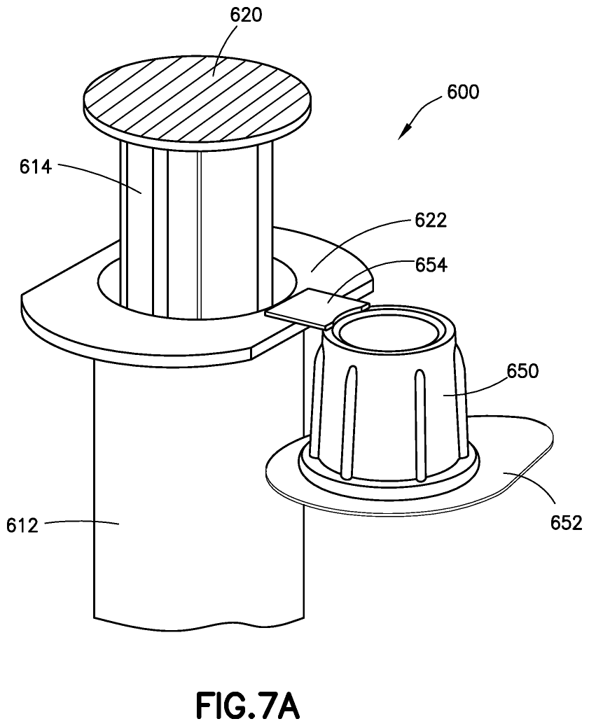

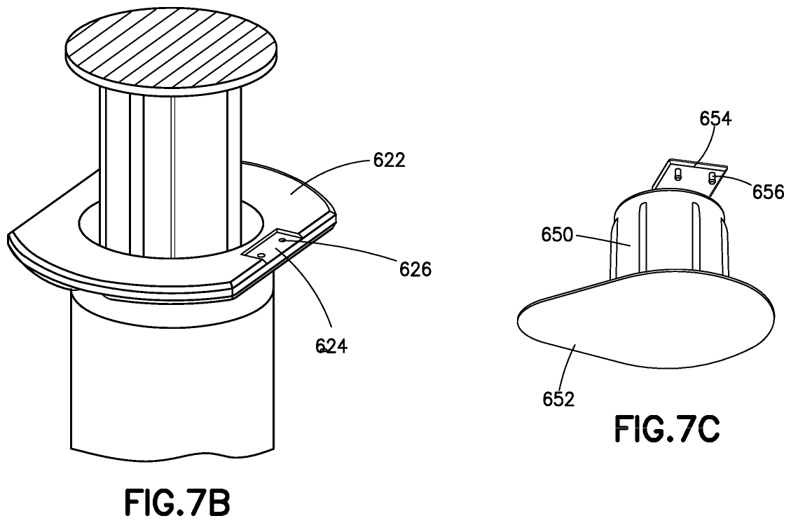

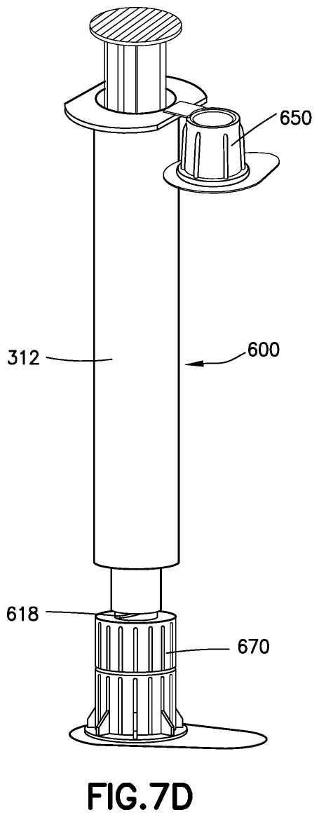

Referring to FIGS. 7A-7D, shown are non-limiting embodiments of pre-filled syringe 600. In some non-limiting embodiments, pre-filled syringe 600 may include barrel 612, plunger rod 614, a stopper, luer connection 618, and thumb press 620. In some non-limiting embodiments, pre-filled syringe 600 may be the same as pre-filled syringe 10 previously described, except that pre-filled syringe 600 includes modified flange 622 as shown in FIGS. 7A-7D.

In some non-limiting embodiments, flange 622 may include slot 624. For example, flange 622 may include slot 624 that is configured to receive plate 654 of cap 650. In some non-limiting embodiments, slot 624 may be recessed into flange 622. In some non-limiting embodiments, slot 624 may have a square cross-section. In some non-limiting embodiments, slot 624 may include at least one opening 626. For example, slot 624 may include at least one opening 626 that extends into flange 622. In some non-limiting embodiments, slot 624 may include at least one opening 626 that correspond to at least one pin 656 on plate 654 of cap 650.

In some non-limiting embodiments, pre-filled syringe system 600 includes cap 650. In some non-limiting embodiments, cap 650 may be the same as cap 150 previously described. In some non-limiting embodiments, cap 650 includes peel film 652. For example, peel film 652 may be the same as peel film 152 previously described. In some non-limiting embodiments, cap 650 may include plate 654. For example, cap 650 may include plate 654 that is attached to outer surface of cap 650, for example on a proximal end (e.g., opposite of film 652) thereof. In some non-limiting embodiments, plate 654 may include at least one pin 656. For example, plate 654 may include at least one pin 656 that protrude out from the surface of plate 654. In some non-limiting embodiments, plate 654 may include at least one pin 656 that correspond to at least one hole 626 of slot 624 of flange 622. In some non-limiting embodiments, cap 650 may be coupled to pre-filled syringe 610. For example, cap 650 may be coupled to pre-filled syringe 610 by inserting the at least one pin 656 into the at least one hole 626 and, optionally with an interference and/or press fit, inserting plate 654 into slot 624. In some non-limiting embodiments, the at least one pin 656 and the at least one hole 626 may form a press fit, where the diameter of hole 626 is slightly smaller than the outer diameter of the at least one pin 656. In some non-limiting embodiments, cap 650 may be removed from pre-filled syringe 600 by pulling cap 650 away from pre-filled syringe 600 such that the at least one pin 656 is pulled out of the at least one hole 626 and plate 654 is pulled out of slot 624.

Referring to FIG. 7D, in some non-limiting embodiments, pre-filled syringe 600 may include scrubbing device 670. In some non-limiting embodiments, scrubbing device 670 may be the same as scrubbing device 170 previously described. In some non-limiting embodiments, scrubbing device 670 may be coupled to (e.g., attached to, connected to, and/or the like) pre-filled syringe 610. In some non-limiting embodiments, scrubbing device 670 may be connected to pre-filled syringe 610 via luer connection 618.

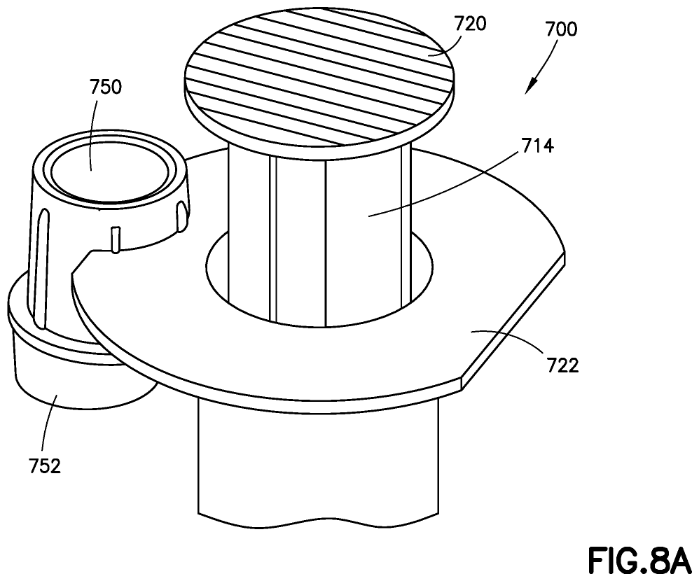

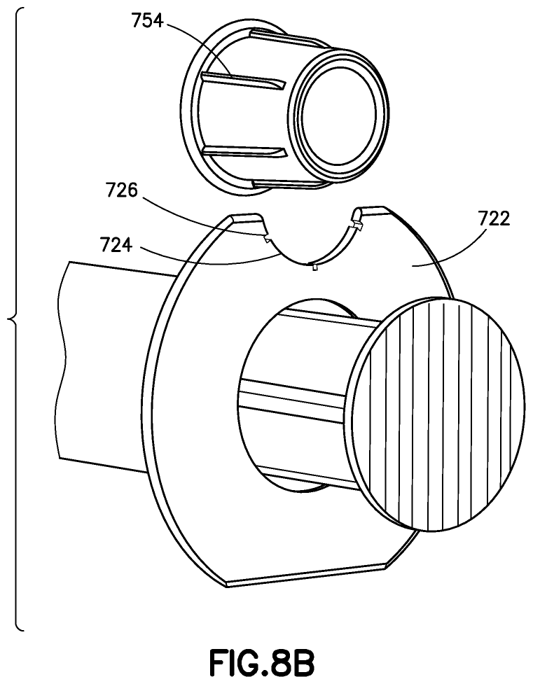

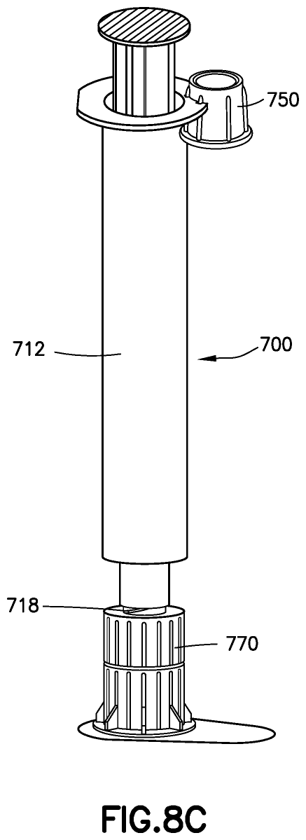

Referring to FIGS. 8A-8C, shown are non-limiting embodiments of pre-filled syringe 700. In some non-limiting embodiments, pre-filled syringe 700 may include barrel 712, plunger rod 714, a stopper, luer connection 718, and thumb press 720. In some non-limiting embodiments, pre-filled syringe 700 may be the same as pre-filled syringe 10 previously described, except that pre-filled syringe 700 includes modified flange 722 as shown in FIGS. 8A-8C.

In some non-limiting embodiments, flange 722 may include recess 724. For example, flange 722 may include recess 724 that is a cut-out portion of flange 722. In some non-limiting embodiments, recess 724 may be a semi-circle cut-out of flange 722. In some non-limiting embodiments, recess 724 may include at least one slot 726 arranged and/or extending radially about cut-out, for example as shown in FIG. 8B. For example, recess 724 may include at least one slot 726 along the cut-out portion of flange 722 that extend into flange 722. In some non-limiting embodiments, recess 724 may include at least one slot 726 that corresponds to at least one rib 754 of cap 750.

In some non-limiting embodiments, pre-filled syringe 700 includes cap 750. In some non-limiting embodiments, cap 750 may be the same as cap 150 previously described. In some non-limiting embodiments, cap 750 includes peel film 752. For example, peel film 752 may be the same as peel film 152 previously described. In some non-limiting embodiments, cap 750 may include at least one rib 754. For example, cap 750 may include at least one rib 754 on the outer surface of cap 750. In some non-limiting embodiments, the at least one rib 754 may be on the outer surface of cap 750 and in the axial direction. In some non-limiting embodiments, the at least one rib 754 may correspond to the at least one slot 726 of recess 724. For example, the at least one rib 754 may be configured to be coupled to the at least one slot 726 of recess 724. In some non-limiting embodiments, cap 750 may be coupled to pre-filled syringe 700 by coupling the at least one rib 754 to the at least one slot 726 of recess 724. In some non-limiting embodiments, cap 750 may be removed from pre-filled syringe 700 by pulling cap 750 away from pre-filled syringe 700 such that the at least one rib 754 is/are removed from the at least one slot 726.

In some non-limiting embodiments, pre-filled syringe 700 may include scrubbing device 770. In some non-limiting embodiments, scrubbing device 770 may be the same as scrubbing device 170 previously described. In some non-limiting embodiments, scrubbing device 770 may be coupled to (e.g., attached to, connected to, and/or the like) pre-filled syringe 70. In some non-limiting embodiments, scrubbing device 770 may be connected to pre-filled syringe 710 via luer connection 718.

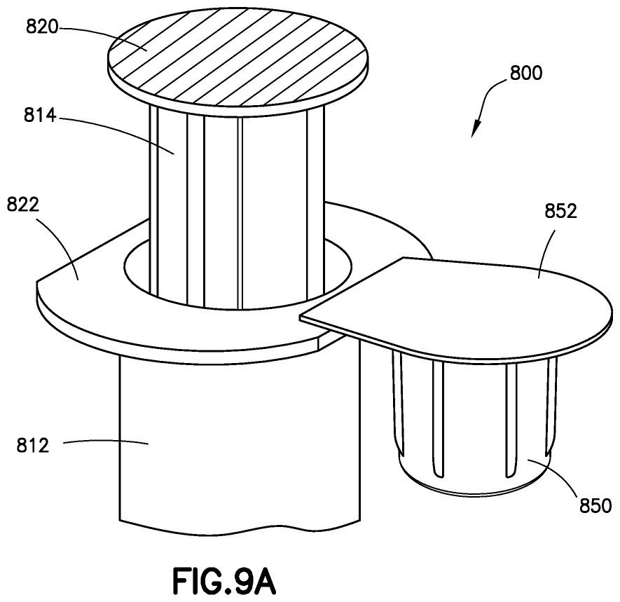

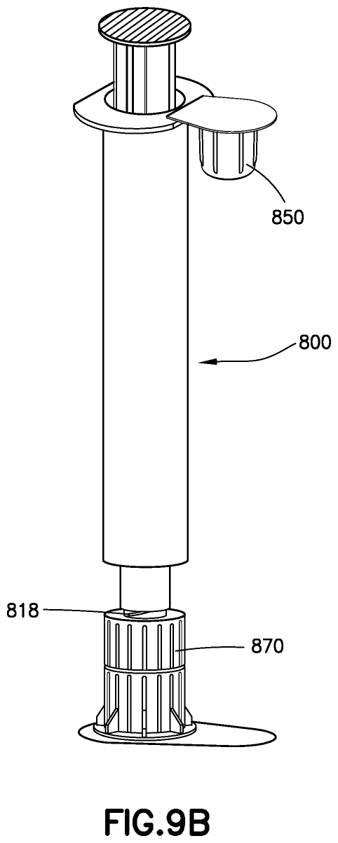

Referring to FIGS. 9A and 9B, shown are non-limiting embodiments of pre-filled syringe 800. In some non-limiting embodiments, pre-filled syringe 800 may include barrel 812, plunger rod 814, a stopper, luer connection 818, and thumb press 820. In some non-limiting embodiments, pre-filled syringe 810 may be the same as pre-filled syringe 10 previously described.

In some non-limiting embodiments, pre-filled syringe 800 includes cap 850. In some non-limiting embodiments, cap 850 may be the same as cap 150 previously described. In some non-limiting embodiments, cap 850 includes peel film 852. For example, peel film 852 may be the same as peel film 152 previously described. In some non-limiting embodiments, peel film 852 may be formed of a heat sealable material. A “heat sealable” material refers to a material that may be heated and applied to a substrate, such that when the material cools, forms a seal with the substrate. In some non-limiting embodiments, cap 850 may be coupled to pre-filled syringe 800. For example, cap 850 may be coupled to pre-filled syringe 800 by heat sealing peel film 852 to flange 822. In some non-limiting embodiments, cap 850 may be removed from pre-filled syringe 800 (e.g., flange 822) by pulling cap 850 away from pre-filled syringe 800 and breaking the heat seal between peel film 852 and flange 822.

Referring to FIG. 9B, in some non-limiting embodiments, pre-filled syringe 800 may include scrubbing device 870. In some non-limiting embodiments, scrubbing device 870 may be the same as scrubbing device 170 previously described. In some non-limiting embodiments, scrubbing device 870 may be coupled to (e.g., attached to, connected to, and/or the like) pre-filled syringe 800. In some non-limiting embodiments, scrubbing device 870 may be connected to pre-filled syringe 800 via luer connection 818.

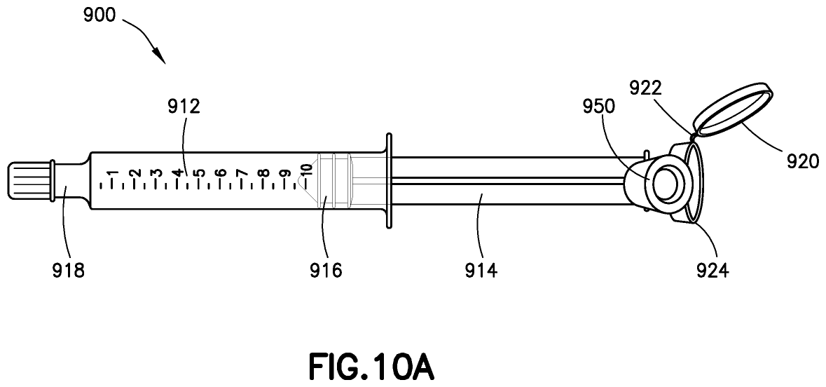

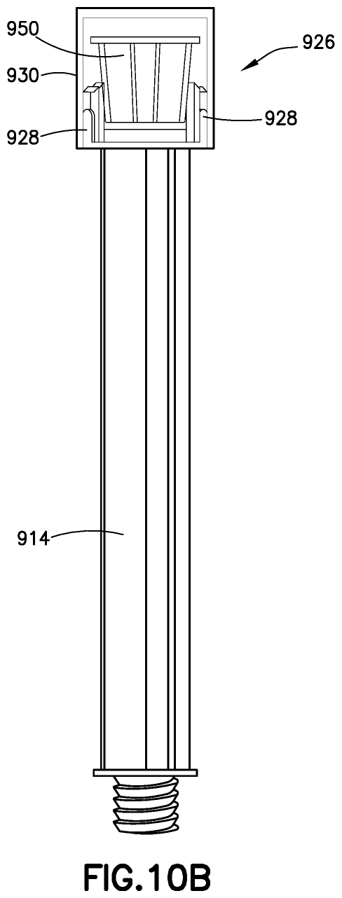

Referring to FIGS. 10A and 10B, shown are non-limiting embodiments of pre-filled syringe 900. In some non-limiting embodiments, pre-filled syringe 900 may include barrel 912, plunger rod 914, stopper 916, luer connection 918, and thumb press 920. In some non-limiting embodiments, pre-filled syringe 900 may be the same as pre-filled syringe 10 previously described, except that plunger rod 914 and thumb press 920 may be modified as shown in FIG. 10A.

In some non-limiting embodiments, plunger rod 914 may include internal volume 924. For example, plunger rod 914 may include internal volume 924 that is configured to hold cap 950. In some non-limiting embodiments, plunger rod 914 may include thumb press 920 that is attached to plunger rod 914 with hinge 922. For example, plunger rod 914 may include thumb press 920 that is attached to plunger rod 914 with hinge 922 and is configured to couple to plunger rod 914 to seal internal volume 924 of plunger rod 914.

In some non-limiting embodiments, pre-filled syringe system 900 includes cap 950. In some non-limiting embodiments, cap 950 may be the same as cap 150 previously described. In some non-limiting embodiments, cap 950 may be contained within internal volume 924 of plunger rod 914, optionally at a proximal end thereof. For example, cap 950 may be contained within internal volume 924 of plunger rod 914 and sealed within internal volume 924 by the coupling (e.g., a hinged coupling, snap coupling, friction fit, or the like) of thumb press 920 and the plunger rod 914. In some non-limiting embodiments, cap 950 may be removed from pre-filled syringe 900 by opening the thumb press 920 and decoupling thumb press 920 from the plunger rod 914 such that cap 950 may be removed from internal volume 924 of plunger rod 914.

In some non-limiting embodiments, as shown in FIG. 10B, pre-filled syringe 900 may include housing unit 926 on plunger rod 914, optionally at a proximal end thereof, as an alternative to the internal volume 924 shown in FIG. 10A. In some non-limiting embodiments, housing unit 926 may include at least one press-in joint 928. For example, housing unit 926 may include two press-in joints 928, or even more. In some non-limiting embodiments, housing unit 926 may include cap cover 930. Cap cover 930 may include at least one press-in joint slot on the internal surface of cap cover 930. In some non-limiting embodiments, the at least one press-in joint slot of the cap cover 930 may be configured to be coupled with at least one press-in joint 928. For example, the number of press-in joint slots of the cap cover 930 may correspond to the number of press-in joints 928. In some non-limiting embodiments, cap 950 may be contained within housing unit 926 of plunger rod 914. For example, cap 950 may be contained within housing unit 926 of plunger rod 914 and sealed within housing unit 926 by the coupling of the at least one press-in joint slot of cap cover 930 with at least one press-in joint 928. In some non-limiting embodiments, cap 950 may be removed from pre-filled syringe 900 by pressing at least one press-in joint 928 to remove at least one press-in joint 928 from the at least one press-in joint slot of the cap cover 930 and decoupling cap cover 930 from housing unit 926 such that cap 950 may be removed from housing unit 926.

In some non-limiting embodiments, pre-filled syringe 900 may include a scrubbing device. In some non-limiting embodiments, the scrubbing device may be the same as scrubbing device 170 previously described. In some non-limiting embodiments, the scrubbing device may be coupled to (e.g., attached to, connected to, and/or the like) pre-filled syringe 900. In some non-limiting embodiments, the scrubbing device may be connected to pre-filled syringe 900 via a luer connection.

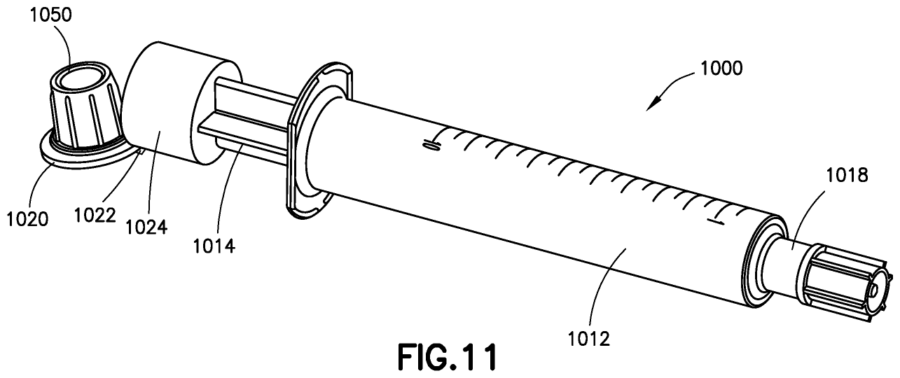

Referring to FIG. 11, shown is a non-limiting embodiment of pre-filled syringe 1000. In some non-limiting embodiments, pre-filled syringe 1000 may include barrel 1012, plunger rod 1014, a stopper, luer connection 1018, and thumb press 1020. In some non-limiting embodiments, pre-filled syringe 1010 may be the same as pre-filled syringe 10 previously described, except that plunger rod 1014 and thumb press 1020 may be modified as shown in FIG. 11.

In some non-limiting embodiments, plunger rod 1014 may include internal volume 1024, optionally arranged at proximal end thereof. For example, plunger rod 1014 may include internal volume 1024 that is configured to hold cap 1050. In some non-limiting embodiments, plunger rod 1014 may include thumb press 1020 that is attached to plunger rod 1014 with hinge 1022. For example, plunger rod 1014 may include thumb press 1020 that is attached to plunger rod 1014 with hinge 1022 and is configured to couple to plunger rod 1014 to seal internal volume 1024 of plunger rod 1014.

In some non-limiting embodiments, pre-filled syringe 1000 includes cap 1050. In some non-limiting embodiments, cap 1050 may be the same as cap 150 previously described. In some non-limiting embodiments, cap 1050 may be contained within internal volume 1024 of plunger rod 1014. For example, cap 1050 may be contained within internal volume 1024 of plunger rod 1014 and sealed within internal volume 1024 by the coupling of thumb press 1020 and the plunger rod 1014. In some non-limiting embodiments, cap 1050 may be coupled to thumb press 1020. For example, cap 1050 may be coupled (e.g., attached) to the inner surface of thumb press 1020 such that when thumb press 1020 is closed and coupled to plunger rod 1014, cap 1050 is contained within internal volume 1024 of plunger rod 1014. In some non-limiting embodiments, cap 1050 may be removed from pre-filled syringe 1010 by opening the thumb press 1020 and decoupling thumb press 1020 from the plunger rod 1014 such that cap 1050 may be removed from internal volume 1024 of plunger rod 1014. In such an embodiment, when the thumb press 1020 is decoupled from plunger rod 1014 to open internal volume 1024, cap 1050 that is attached to the inner surface of thumb press 1020 is removed from internal volume 1024 of plunger rod 1014.

In some non-limiting embodiments, pre-filled syringe 1000 may include a scrubbing device. In some non-limiting embodiments, the scrubbing device may be the same as scrubbing device 170 previously described. In some non-limiting embodiments, the scrubbing device may be coupled to (e.g., attached to, connected to, and/or the like) pre-filled syringe 1010. In some non-limiting embodiments, the scrubbing device may be connected to pre-filled syringe 1010 via luer connection 1018.

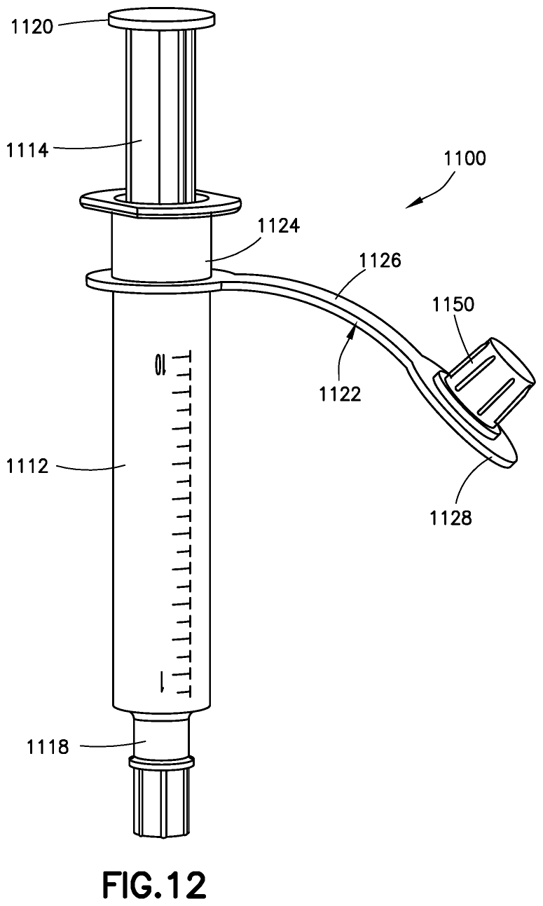

Referring to FIG. 12, shown is a non-limiting embodiment of pre-filled syringe 1100. In some non-limiting embodiments, pre-filled syringe 1100 may include barrel 1112, plunger rod 1114, a stopper, luer connection 1118, and thumb press 1120. In some non-limiting embodiments, pre-filled syringe 1100 may be the same as pre-filled syringe 10 previously described.

In some non-limiting embodiments, pre-filled syringe system 1100 includes cap 1150. In some non-limiting embodiments, cap 1150 may be the same as cap 150 previously described.

In some non-limiting embodiments, cap 1150 may be coupled to pre-filled syringe 1110, such as coupled to barrel 1112. For example, cap 1150 may be coupled to pre-filled syringe 1100, such as coupled to barrel 1112, with tether arrangement 1122. In some non-limiting embodiments, tether arrangement 1122 may include first tether ring 1124. First tether ring 1124 may be positioned around the outer surface of barrel 1112 to couple tether arrangement 1122 to barrel 1112. In some non-limiting embodiments, first tether ring 1124 may form a part of barrel 1112. In some non-limiting embodiments, first tether ring 11244 may be removable from barrel 1112. In some non-limiting embodiments, tether arrangement 1122 may include elongated portion 1126. For example, tether arrangement 1122 may include elongated portion 1126 that attaches first tether ring 1124 to second tether ring 1128. In some non-limiting embodiments, tether arrangement 1124 may include second tether ring 1128. In some non-limiting embodiments, cap 1150 may be coupled to second tether ring 1126. For example, cap 1150 may be attached to (e.g., adhered to and/or the like) second tether ring 1126. In some non-limiting embodiments, cap 1150 may be removed from pre-filled syringe 1100 by removing (e.g., pulling and/or the like) cap 1150 from tether arrangement 1122. For example, cap 1150 may be removed from pre-filled syringe 1100 by removing (e.g., pulling and/or the like)) cap 1150 from second tether ring 1128. In some non-limiting embodiments, cap 1150 may be removed from pre-filled syringe system 1100 by removing tether arrangement 1122 from barrel 1112 and then removing cap 1150 from tether arrangement 1122. For example, cap 1150 may be removed from pre-filled syringe 1100 by removing tether arrangement 1122 from barrel 1112 and then removing cap 1150 from second tether ring 1128.

In some non-limiting embodiments, pre-filled syringe 1100 may include a scrubbing device. In some non-limiting embodiments, the scrubbing device may be the same as scrubbing device 170 previously described. In some non-limiting embodiments, the scrubbing device may be coupled to (e.g., attached to, connected to, and/or the like) pre-filled syringe 1100. In some non-limiting embodiments, the scrubbing device may be connected to pre-filled syringe 1100 via luer connection 1118.

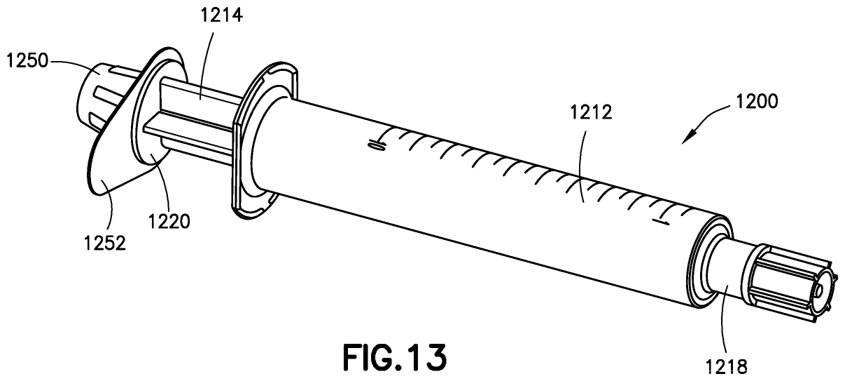

Referring to FIG. 13, shown is a non-limiting embodiment of pre-filled syringe 1200. In some non-limiting embodiments, pre-filled syringe 1200 may include barrel 1212, plunger rod 1214, a stopper, luer connection 1218, and thumb press 1220. In some non-limiting embodiments, pre-filled syringe 1210 may be the same as pre-filled syringe 10 previously described.

In some non-limiting embodiments, pre-filled syringe system 1200 includes cap 1250. In some non-limiting embodiments, cap 1250 may be the same as cap 150 previously described. In some non-limiting embodiments, cap 1250 includes peel film 1252. For example, peel film 1252 may be the same as peel film 152 previously described.

In some non-limiting embodiments, cap 1250 may be coupled to thumb press 1220. For example, peel film 1252 may be coupled to thumb press 1220. In some non-limiting embodiments, peel film 1252 may be attached to thumb press 1220. For example, peel film 1252 may be adhered to thumb press 1220. In another example, peel film 1252 may be heat sealed to thumb press 1220. In some non-limiting embodiments, cap 1250 may be removed from thumb press 1220. For example, cap 12500 may be removed from thumb press 1220 by peeling cap 1250 off of thumb press 1220.

In some non-limiting embodiments, pre-filled syringe 1200 may include a scrubbing device. In some non-limiting embodiments, the scrubbing device may be the same as scrubbing device 170 previously described. In some non-limiting embodiments, the scrubbing device may be coupled to (e.g., attached to, connected to, and/or the like) pre-filled syringe 1200. In some non-limiting embodiments, the scrubbing device may be connected to pre-filled syringe 1200 via luer connection 1218.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A pre-filled syringe, comprising:
a barrel comprising:
a proximal end, a distal end, and a sidewall extending between the proximal and distal end, the sidewall defining a chamber that contains a medical solution;
a plunger rod comprising a proximal end and a distal end;
a stopper attached to the distal end of the plunger rod and moveably received in the chamber; and
a disinfecting cap releasably coupled to the pre-filled syringe, the disinfecting cap comprising a housing defining an interior configured to hold a disinfecting solution.

2. The pre-filled syringe of claim 1, wherein the plunger further comprises:
a thumb press at the proximal end of the plunger rod; and
a socket positioned between the plunger rod and the thumb press of the plunger;
wherein the disinfecting cap is contained within the socket.

3. The pre-filled syringe of claim 1, wherein the plunger further comprises:
a thumb press at the proximal end of the plunger rod; and
a cap holder comprising a lock and positioned between the plunger rod and the thumb press,
wherein the disinfecting cap comprises a pin; and
wherein the pin is configured to releasably couple with the lock such that the cap can be removed from the cap holder.

4. The pre-filled syringe of claim 1, wherein the plunger further comprises:
a thumb press at the proximal end of the plunger rod; and
a cap holder attached to the thumb press;
wherein the disinfecting cap is received within the cap holder.

5. The pre-filled syringe of claim 1, wherein the barrel further comprises:
a flange at the proximal end thereof, the flange comprising one or more slots; and
wherein the disinfecting cap comprises a guide configured to be positioned within the one or more slots to releasably attach the disinfecting cap to the pre-filled syringe.

6. The pre-filled syringe of claim 1, wherein the disinfecting cap comprises:
a ring comprising a snap connection, wherein the ring is configured to be arranged around an outer surface of the barrel to releasably attach the disinfecting cap to the pre-filled syringe.

7. The pre-filled syringe of claim 1, wherein the barrel comprises:
a flange at the proximal end of the barrel, the flange comprising a slot having one or more openings therein, and
wherein the disinfecting cap comprises a plate comprising at least one pin, wherein the plate is configured to be positioned within the slot such that the at least one pin is positioned within the at least one opening to releasably attach the disinfecting cap to the pre-filled syringe.

8. The pre-filled syringe of claim 1, wherein the barrel comprises:
a flange at the proximal end of the barrel, the flange comprising a recess comprising at least one slot, and
wherein the disinfecting cap comprises at least one rib extending axially on the outer surface of the disinfecting cap, such that the at least one rib is configured to be positioned within the at least one slot to releasably attach the disinfecting cap to the pre-filled syringe.

9. The pre-filled syringe of claim 1, wherein the barrel comprises:
a flange at the proximal end of the barrel; and
wherein the disinfecting cap comprises a peel film that is heat sealed to the flange, thereby attaching the disinfecting cap to the pre-filled syringe.

10. The pre-filled syringe of claim 1, wherein the plunger rod comprises:
an internal volume at the proximal end of the plunger rod; and
a thumb press attached to the internal volume with a hinge,
wherein the disinfecting cap is configured to be positioned within the internal volume of the plunger.

11. The pre-filled syringe of claim 10, wherein the disinfecting cap is configured to be releasably attached to an inner surface of the thumb press.

12. The pre-filled syringe of claim 1, further comprising a tether having a first end and a second end, wherein the first end is attached to the pre-filled syringe and the second end is attached to the disinfecting cap.

13. The pre-filled syringe of claim 1, wherein the plunger rod comprises:
a thumb press at the proximal end of the plunger rod, wherein a peel film of the disinfecting cap is attached to the thumb press.

14. The pre-filled syringe of claim 1, wherein the disinfecting solution comprises an alcohol.

15. The pre-filled syringe of claim 1, wherein the medical solution is a flush solution.

16. The pre-filled syringe of claim 1, wherein the medical solution is a catheter lock solution.

17. The pre-filled syringe of claim 1, wherein the barrel comprises a luer connector at the distal end thereof.

18. The pre-filled syringe of claim 17, further comprising a scrubbing device releasably coupled to the luer, the scrubbing device comprising a housing defining an interior configured to hold a disinfecting solution.

19. The pre-filled syringe of claim 18, wherein the disinfecting solution comprises an alcohol.
